(19) United States
(12) Patent Application Publication
(10) Pub. No.: US 2005/0090791 A1
(43) Pub. Date: Apr. 28, 2005

(54) Shock-absorbing and sterile body hygiene article such as a panty-napkin

(76) Inventor: Tahar Jerou, Vernouillet (FR)

Correspondence Address: YOUNG & THOMPSON, 745 SOUTH 23RD STREET, 2ND FLOOR, ARLINGTON, VA, 22202, US

(21) Appl. No.: 10/503,634

(22) PCT Filed: Feb. 3, 2003
(86) PCT No.: PCT/FR03/00323

(30) Foreign Application Priority Data
Feb. 2, 2002 (FR) 0201574

(51) Int. Cl. A61F 13/15; A61F 13/20

(52) U.S. Cl. 604/385.03

(57) ABSTRACT

A body hygiene article such as, for example, an infant's panty-napkin adapted to protect the wearer's genital system, includes a soft and impermeable outer sheet (2) and an absorbent layer (8, 9) arranged inside the outer sheet. The article further includes a protective shell (5) permeable to liquids separated from the soft and impermeable outer sheet by at least part (8) of the absorbent layer, and arranged and configured so as to be sufficiently rigid to cover the genital system.

The invention relates to a body hygiene article such as a panty-napkin adapted to protect the genital system, for example following an accident, surgery or even simple irritation. It is aimed in particular, although not exclusively, at a panty-napkin for young boys who have just been circumcised or for injured, ill or infirm persons of either sex. It is aimed equally at a panty-napkin specifically designed for this purpose and at an article intended to co-operate with a conventional panty or panty-napkin for the same purpose. Finally, it is aimed very generally at a body hygiene article adapted to protect any area of the user's body.

The principle of panty-napkin construction is well known in the art. As described in the documents FR-A-2 413 048, FR-A-2 438 434 and FR-A-2 607 366 in particular, a napkin comprises mainly a flexible exterior film that is impermeable to liquids, for example a polyethylene film, an absorbent pad comprising one or more layers inside this flexible layer, for example layers of cellulose wadding, and usually a permeable interior film, conventionally of non-woven material.

Certain traditions involve the circumcision of boys, sometimes at the age of a few weeks or a few months, when the child is not yet able to control its sphincters. The use of a panty-napkin is inappropriate until the scar has healed completely, since in practice there is rubbing and the risk of impacts in the case of movement over the area that is healing, with the result that it is often necessary to dispense temporarily with any panty-napkin, with all the inconveniences that may be imagined.

The same problems may arise after any kind of surgery on the genital system of a baby or even on an incontinent, immobilized or infirm adult man or woman.

Thus a first object of the invention is a panty-napkin that can be used even when the genital system must be protected from impact, and even from mere contact, as well as from the urine of the user.

A second object of the invention is an article adapted to cooperate with a conventional panty-napkin, and even with a conventional panty, to protect the genital system from impact, or even from mere contact, or from the urine of the wearer.

Other areas of the body liable to produce body fluids may benefit from protection against impact or rubbing; this applies in particular to any area in which a scar is healing, and this protection is something that conventional dressings are unable to provide properly.

For this reason a further object of the invention is an article adapted to protect an area of the body from impacts and even from mere contact.

To this end the invention proposes a panty-napkin adapted to protect the wearer's genital system, comprising an exterior flexible and impermeable film and an absorbent layer disposed inside said exterior film, characterized in that it further comprises an anti-impact protective shell permeable to liquids separated from the exterior flexible and impermeable film by at least one portion of the absorbent layer and disposed and conformed in a sufficiently rigid manner to cap the genital system without pressing on it.

According to preferred and where applicable combined features of the invention:
the absorbent layer comprises two layers respectively outside and inside the shell,
the layer inside the shell is thinner than the layer outside it,
the layers are in contact at their edges with an absorbent layer portion intended to pass under the perineum and the buttocks,
the absorbent layer extends under the perineum and the buttocks,
the shell is made from an elastic or perforated plastic material with a plurality of holes,
the shell is made from a braided material,
the shell is made from a coated textile material,
an adhesive area is provided on its interior surface for fixing it to the skin of the wearer,
the shell is lined with a permeable film.

The invention further proposes a hygiene article adapted to co-operate with a panty or a panty-napkin and comprising an exterior flexible and impermeable film and an absorbent layer disposed inside said exterior film, said article comprising an anti-impact protective shell permeable to liquids, provided with fixing means for positioning it in a configuration in which it is separated from the exterior flexible and impermeable film by at least one portion of the absorbent layer, and disposed and conformed in a sufficiently rigid manner to cap the genital-system without pressing on it.

According to preferred features of the invention, according to this second feature:
the article further comprises an inner absorbent layer inside the permeable shell,
the article further comprises a permeable film inside the shell,
the fixing means are adapted to fix the shell to the body,
the fixing means are adapted to fix the shell to the inside of a panty or panty-napkin,
the shell is made from an elastic material or a plastic material perforated with a plurality of holes.

Another aspect of the invention proposes a dressing adapted to protect an area of the body of the wearer, comprising an exterior flexible and impermeable film and an absorbent layer disposed inside this exterior film, characterized in that it further comprises an anti-impact protective shell permeable to liquids, separated from the exterior flexible and impermeable film by at least one portion of the absorbent layer, and disposed and conformed in a sufficiently rigid manner to cap the area to be protected without pressing on it.

It will be noted that the panty-napkin defined hereinabove is a particular instance of this kind of article.

Objects, features and advantages of the invention will emerge from the following description, which is given by way of nonlimiting example and with reference to the appended drawings, in which:

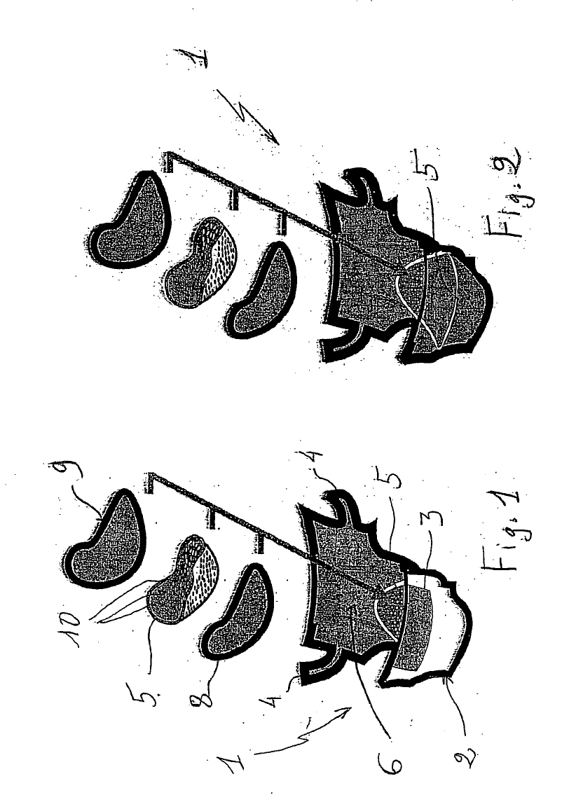

FIG. 1 is a perspective view of a panty-napkin conforming to the invention, the protective portion of this napkin being represented in place and in exploded form,

FIG. 2 is another perspective view of a panty-napkin differing from that of FIG. 1 in that the protective part is visible through the exterior layer of the panty-napkin,

FIG. 3 is a partial view of the panty-napkin in vertical section,

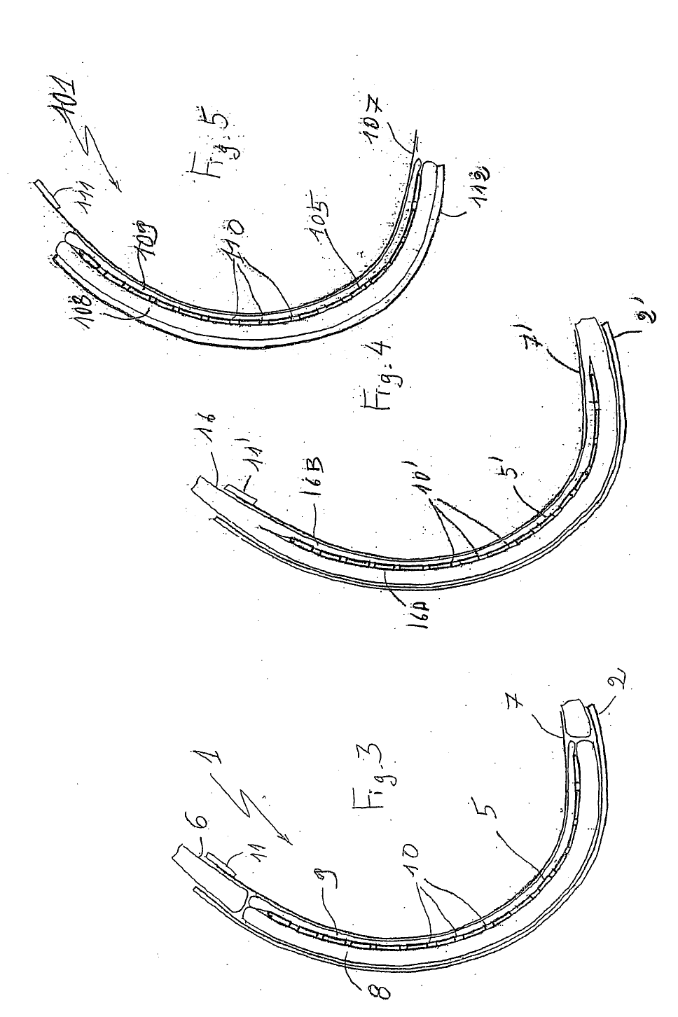

FIG. 4 is a partial view in vertical section of a variant of the panty-napkin from FIGS. 1 to 3, and

FIG. 5 is a part-sectional view of a hygiene article adapted to co-operate with a panty or a panty-napkin to form a combination equivalent to the FIG. 3 napkin.

FIGS. 1 to 3 are general representations of a hygiene article intended to protect an area of the body of a wearer, comprising an exterior flexible and impermeable film and an absorbent layer disposed inside said exterior film, characterized in that it further comprises an anti-impact protective shell permeable to liquids separated from the exterior flexible and impermeable film by at least one portion of the absorbent layer and disposed and conformed in a sufficiently rigid manner to cap the area to be protected without pressing on it.

To be more precise, in the example represented, the article is a panty-napkin 1 comprising, in accordance with the invention, a flexible and impermeable exterior film 2, conventionally provided with complementary fixing areas 3 and 4, one or more layers of absorbent material, and a rigid protective shell 5 that is permeable to liquids, separated from the flexible exterior film by one or more portions of the absorbent material layer, and intended to cover the genital system of the user of the panty-napkin 1.

Outside the area comprising this shell facing the genital system, the panty-napkin 1 has a conventional configuration, for example with an additional absorbent material layer 6 intended in particular to pass under the perineum and the buttocks of the user, and elastic areas (not shown) intended to make the panty-napkin comfortable to wear. These features being characteristic, they are not described in detail here. In practice the absorbent layer is joined to the complementary layer 6, and the flexible permeable film 7 (see FIG. 3) provides a lining of the panty-napkin, at least in the areas thereof provided with absorbent material.

In the example represented, the absorbent material layer comprises two layers 8 and 9 disposed outside and inside the shell 5, respectively.

The shell 5 being permeable, it allows any liquid to flow from the interior layer 9 to the layer 8, helping to minimize the wetness of the layer 9 nearest the genital system.

To encourage storage of liquid outside the shell, the exterior layer 8 is preferably thicker than the interior layer 9. In a variant that is not shown the layer 8 is omitted, but its presence is advantageous in that it contributes to the comfort of the user and allows rapid absorption of liquid when it appears.

The permeable shell is intended to protect the genital system from untimely contact, pressure or impact during use of the panty-napkin, whether in bed (for example on turning over) or during activities that are in principle calm: thus the shell does not need to be as rigid as the shells used to protect the genital system during rough or combat sports, for example. The shell 5 is rigid in the sense that it has a concave configuration adapted to cap the genital system of the male or female person and is able to retain this configuration for the time of use of a panty-napkin, i.e. for just a few hours.

Thus the shell 5 may be much thinner and weaker than sports shells. It is made from a plastic material such as propylene, for example, or an elastic material such as rubber, and is a few millimeters thick.

In the example shown, the shell 5 is made from an impermeable plastic material and is made permeable by many perforations 10, which are preferably well distributed over the whole of the surface of the shell.

Given the generally filamentary nature of the non-woven absorbent material that constitutes the exterior layer 8 and the interior layer 9 (for example cellulose wadding), these perforations may serve to bring the filaments of each of the layers into contact, which facilitates drainage of the liquid from the interior of the shell to the exterior.

In a variant that is not shown, the shell may be formed of a woven or non-woven textile material stiffened by coating it so that it is able to retain its shape for several hours, without the shape degrading too quickly when a discharge of liquid passes through it. In this case, the shell is of permeable construction. Other configurations are feasible; thus the shell may be produced by braiding plastic or elastic filaments, etc. In fact, as just explained, the permeability of the shell may result from the intrinsic permeability of the material from which it is made or from the geometry with which that material is configured (for example, braided, perforated, etc.).

Of course, the person skilled in the art knows how to define the geometry (shape and contour) of the shell so that its presence in use is not uncomfortable, as a function of the age and the sex of the intended wearer of the panty-napkin.

In the example represented, the layers 8 and 9 have the shell sandwiched between them and have substantially the same contour (these layers are preferably slightly larger than the shell to ensure that the liquid may be assimilated over the whole of the surface of the shell, including along its edges). The edge of these layers (or the edge of the exterior layer if there is no interior layer) extends as far as contact with the layer 6, to ensure good continuity of pumping of the liquid.

In the FIG. 4 embodiment, the exterior and interior layers are in fact extensions, in the form of two sub-layers 16A and 16B, of the massive layer 16 provided in areas other than facing the genital system. The panty-napkin differs from that of FIGS. 1 to 3 only in this regard and is not described in more detail. In this figure, items analogous to those from FIGS. 1 to 3 have the same reference symbols as FIGS. 1 to 3 but “primed”.

The permeable flexible film 7 or 7′, when present, is advantageously stuck to the interior of the internal absorbent layer 9 or 16B, or even directly to the shell. It faces the body area to be protected.

In practice, the conventional ways of fixing panty-napkins offer sufficient performance to achieve good retention. Nevertheless, to guarantee good retention of the shell, even in the event of movement, the panty-napkin of the invention preferably and advantageously comprises, on its interior surface, an adhesive area 11 or 11′ for temporary adhesion to the body of the user, for example just above the genital system.

FIG. 5 represents an article which, with a conventional panty-napkin (or impermeable panty), not shown, produces the FIG. 3 configuration.

In FIG. 5, items analogous to those from FIG. 3 are represented by the same reference numbers plus 100.

Thus this article comprises an anti-impact protective shell 105 that is permeable to liquids and provided with means 111 for fixing it to the skin adapted to position the shell over a body area of a wearer in a configuration in which it is able to cap that area without pressing on it.

Thus when this article is worn with a panty-napkin or an impermeable panty, a configuration is obtained in which the shell is separated from the exterior flexible and impermeable film of the panty or panty-napkin by an absorbent layer that is preferably part of the panty or panty-napkin.

However, as shown here, this article advantageously comprises an absorbent layer 110 fixed to the external face of the shell to drain to the exterior fluids that may be generated inside the shell.

It is also preferable if this article comprises an internal absorbent layer 109 lining the shell and improving fluid absorption and drainage.

For comfort's sake, independently of the presence of the layer 109, there is advantageously a flexible permeable film 107 facing the body portion to be protected.

In the example represented, the fixing means 111 are adhesive areas intended to be stuck to the skin and fixed to the film 107, although in a variant that is not shown they may also be fixed to a projecting portion of the external absorbent layer, as in the case of a conventional dressing, which guarantees good retention of the shell. These means 111 for fixing to the skin may also be fixed to the internal layer 109 or to the shell 105 itself.

In that the article is intended for single use, its retention is not necessarily of the same quality as that of a dressing.

As an alternative, the article may also be provided with means for indirect fixing to the body, i.e. means for fixing to the panty or panty-napkin inside which the article is intended to be worn, this panty or panty-napkin being provided with its usual dedicated means for retention on the body (i.e. the conventional adhesive tabs of a panty-napkin or the part of a panty forming a waistband).

Here the indirect fixing means are represented in the form of an adhesive layer 112, for example covered with a removable protective film (not shown) before it is used. The means 112 may instead be fixed to any other portion of the article (internal layer 109, film 107, or even the shell 105 itself).

The means 111 and 112 may be combined.

The adhesive of the means 111 is advantageously anti-allergenic.

It is clear that the FIG. 3 configuration and the FIG. 4 configuration are of very general application, for example to cover a body area other than the genital system, the concavity of the shell being able to cap at a distance, and thus without contact, an area to be protected, for example a wound that is healing and may generate fluids that have to be evacuated from the wound as much as possible.

It goes without saying that the foregoing description has been given by way of non-limiting illustrative example only and that numerous variants may be proposed that do not depart from the scope of the invention.

1. Body hygiene article to protect an area of the body of the wearer, comprising an exterior flexible and impermeable film (2, 2′) and an absorbent layer (8, 9, 16A, 16B) disposed inside this exterior film, characterized in that it further comprises an anti-impact protective shell (5, 5′) permeable to liquids, separated from the exterior flexible and impermeable film by at least one portion (8, 16A) of the absorbent layer, and disposed and conformed in a sufficiently rigid manner to cap the area to be protected without pressing on it.

2. Article according to claim 1, forming a panty-napkin adapted to protect the genital system of the wearer.

3. Article according to claim 1, characterized in that the absorbent layer comprises two layers (8, 9, 16A, 16B) respectively outside and inside the shell.

4. Article according to claim 3, characterized in that the layer inside the shell is thinner than the layer outside it.

5. Article according to claim 3, characterized in that the layers (8, 9) are in contact at their edges with an absorbent layer portion (6) intended to pass under the perineum and the buttocks.

6. Article according to claim 1, characterized in that the absorbent layer (16A, 16B) extends under the perineum and the buttocks.

7. Article according to claim 1, characterized in that the shell (5, 5′) is made from an elastic or perforated plastic material with a plurality of holes (10, 10′).

8. Article according to claim 1, characterized in that the shell is made from a braided material.

9. Article according to claim 1, characterized in that the shell is made from a coated textile material.

10. Article according to claim 1, characterized in that an adhesive area (11, 11′) is provided on its interior surface for fixing it to the skin of the wearer.

11. Article according to claim 1, characterized in that the shell is lined with a permeable film (7, 7′).

12. Hygiene article adapted to co-operate with a panty or a panty-napkin and comprising an exterior flexible and impermeable film and an absorbent layer disposed inside said exterior film, said article comprising an anti-impact protective shell permeable to liquids, provided with fixing means (111, 112) for positioning it in a configuration in which it is separated from the exterior flexible and impermeable film by at least one portion of the absorbent layer, capping the genital system, the shell being conformed in a sufficiently rigid manner to cap the genital system without pressing on it.

13. Hygiene article according to claim 12, characterized in that it comprises an absorbent layer (108) outside the shell.

14. Hygiene article according to claim 12, characterized in that it comprises an absorbent layer (109) inside the shell.

15. Hygiene article according to claim 12, characterized in that it comprises a flexible permeable film facing the genital system.

16. Hygiene article according to claim 12, characterized in that the fixing means comprise adhesive areas (111) adapted to stick to the skin.

17. Hygiene article according to claim 12, characterized in that the fixing means comprise adhesive areas (112) adapted to stick to the panty or panty-napkin.

18. Article according to claim 2, characterized in that the absorbent layer comprises two layers (8, 9, 16A, 16B) respectively outside and inside the shell.

19. Article according to claim 4, characterized in that the layers (8, 9) are in contact at their edges with an absorbent layer portion (6) intended to pass under the perineum and the buttocks.

20. Article according to claim 2, characterized in that the absorbent layer (16A, 16B) extends under the perineum and the buttocks.
